(19) United States
(12) Patent Application Publication
(10) Pub. No.: US 2020/0113456 A1
(43) Pub. Date: Apr. 16, 2020

(54) Securing and Positioning Device for Pressure Transducers

(71) Applicant: Taylor Ross Tucker, Greenville, SC (US)

(72) Inventor: Taylor Ross Tucker, Greenville, SC (US)

(21) Appl. No.: 16/598,645

(22) Filed: Oct. 10, 2019

Related U.S. Application Data
(60) Provisional application No. 62/745,301, filed on Oct. 13, 2018.

(51) Int. Cl. A61B 5/021; A61B 5/00

(52) U.S. Cl. CPC A61B 5/02141; A61B 2562/0247; A61B 5/6823; A61B 5/6833

(57) ABSTRACT

A pressure transducer securing device includes an adhesive pad securable to a patient at a phlebostatic axis of the patient. An adapter is coupled to the adhesive pad. The adapter is configured for receipt of a pressure transducer in order to mount the pressure transducer to the patient. The adapter is fixed or swivalable relative to the adhesive pad.

### CROSS-REFERENCE TO RELATED APPLICATION

The present application is related and has right of priority to U.S. Provisional Patent Application No. 62/745,301 of Tucker filed on Oct. 13, 2018 and entitled “Novel Securing and Positioning Device for Pressure Transducers and the Like,” the entirety of which is incorporated by reference for all purposes.

### FIELD OF THE INVENTION

The present subject matter relates generally to pressure transducer securement devices and apparatuses for positioning and securing pressure transducers to a patient. More particularly, the invention relates to a securement device that is capable of positioning and securing pressure transducers of various sizes and shapes to a phlebostatic axis of a patient.

### BACKGROUND OF THE INVENTION

Critical, hemodynamically unstable patients frequently require accurate pressure readings when previous blood pressure readings were questionable or unattainable through other non-invasive means. More accurate pressure readings are attainable through invasive intravascular catheters transmitted through pressure transducers to attain arterial, central venous, and pulmonary artery pressures. Multiple manufacturers design and develop proprietary pressure transducers to measure the aforementioned vascular pressures.

Obtaining accurate pressure readings poses challenges. A key element to obtaining accurate pressure readings, once the intravascular pressure line has been established, is the location of the pressure transducer relative to the patient. The pressure transducer, no matter the manufacturer, must remain at the same location in relation to the patient to obtain accurate pressure readings. This location on the patient is called the phlebostatic axis, which is located at the patient's mid-axillary line and fourth (4^{th}) intercostal space, level with the heart.

Various clips and holders are available to fit corresponding pressure transducers to a patient's bed or bedside intravenous (IV) pole and level with the phlebostatic axis. A major drawback to affixing the pressure transducers to the bed pole or a bedside IV pole is every time the patient is moved for procedures or scans, the transducer requires re-leveling with the phlebostatic axis and re-zeroing on the monitor to recalibrate accurate pressure readings. Certain securing devices have been created and designed to affix to a patient's arm; however, as mentioned previously, every time the arm is moved away from the phlebostatic axis, the transducer requires re-leveling and re-zeroing and recalibrating.

Many times in emergent, critical situations or when a patient is in transport, pressure transducer clips designed to attach to the bed or IV pole are not readily available. In these scenarios, the pressure transducers are simply taped to the patient's chest wall at the phlebostatic axis as a temporary solution and quick work around. The drawback to simply taping to pressure transducers to the patient is, in many instances, the tape fails to properly secure the transducer at the proper location, and it is difficult to tape the transducer directly to the patient given the functionality and shape of the transducers.

### BRIEF DESCRIPTION OF THE INVENTION

In view of the above, a need exists for a pressure transducer securing device, which affixes directly to the patient at the phlebostatic axis. This transducer securing device eliminates the need for quick fixes, when clips are unavailable, and prevents the need to frequently re-level, re-zero, and recalibrate the transducer each time the patient is moved that is problematic with known clips and holders. Aspects and advantages of the invention will be set forth in part in the following description, or may be apparent from the description, or may be learned through practice of the invention.

In an example embodiment, a pressure transducer securement device generally includes of an adherent backed pad, which is configured for securing to the phlebostatic axis of a patient and conforming the pressure transducer to patient position changes. Interchangeable or fixed adapters with a universal clasp may be used to affix varying manufacturer pressure transducers currently on the market. The pressure transducer securement adapters may be configured to house a single, double, or triple pressure transducer, as needed. In preferred example embodiments, the pressure transducer securement device may be configured with a fixed, swiveling or ratcheting adapter and a universal clasp, which is able to adhere and maintain the position of each pressure transducer relative to the phlebostatic axis of the patient.

In an example embodiment, a pressure transducer securing device includes an adhesive pad securable to a patient at a phlebostatic axis of the patient. An adapter is coupled to the adhesive pad. The adapter is configured for receipt of a pressure transducer in order to mount the pressure transducer to the patient. The adapter is fixed or swivalable relative to the adhesive pad.

In an example embodiment, a method for securing a pressure transducer to a patient includes securing an adhesive pad to the patient at a phlebostatic axis of the patient, and sliding the pressure transducer into an adapter coupled to the adhesive pad. The adapter is fixed or swivalable relative to the adhesive pad.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 is a side view a supine patient with a pressure transducer securement device according to an example embodiment positioned on the supine patient at the phlebostatic axis of the patient.

FIG. 2 is a top, plan view the example pressure transducer securement of FIG. 1.

FIG. 3 is a side, elevation view the example pressure transducer securement of FIG. 1.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the term “and/or” includes any and all combinations of one or more of the associated listed items. As used herein, the singular forms “a,” “an,” and “the,” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the relevant art. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Pressure transducer securing devices, apparatuses, and methods for positioning pressure transducers at the phlebostatic axis are discussed herein. In the following description, for purposes of explanation, numerous specific details are set forth in order to provide a thorough understanding of the present invention. It will be evident, however, to one skilled in the science that the present invention may be practiced without these specific details.

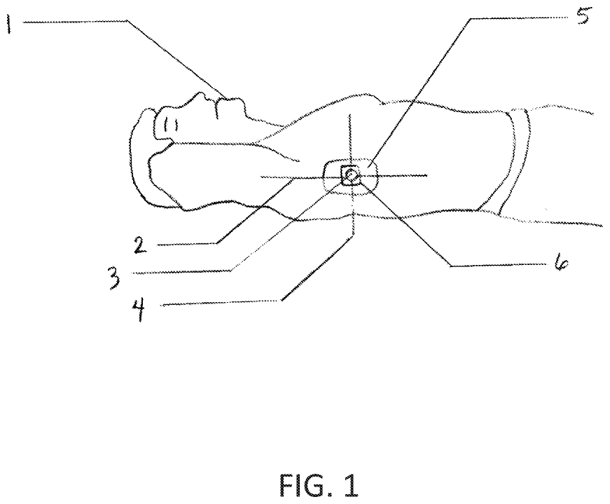

FIG. 1 depicts a side, profile view of a patient 1 lying supine displaying the proper placement of a pressure transducer within a securing device (the “device”) according to various example embodiments of the present invention. In preferred example embodiments, the device may include an adhesive backed pad 5 and a pressure transducer adapter (“adapter”) 6. The phlebostatic axis 3 is located at the midaxillary line 2 and fourth (4^{th}) intercostal space 4 of the patient 1. In preferred example embodiments, the device adheres to the patient 1 at the phlebostatic axis 2 via an adhesive backed pad 5 and includes an adapter 6 capable of securing various pressure transducers to the proper position of the patient 1, in order to obtain accurate pressure readings.

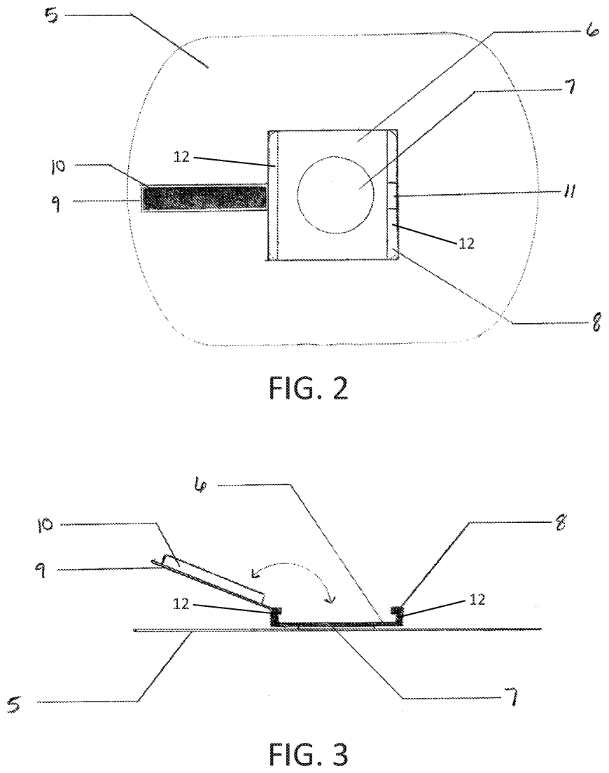

FIG. 2 is a top, plan view of the device. In preferred example embodiments, the device includes an adhesive backed pad 5, on which a pressure transducer adapter (“adapter”) 6 is mounted. In some example embodiments, the device may include a fixed, swiveling, or ratcheting adapter 6. In some example embodiments, the device may be configured with a single, double, or triple adapter 6. Thus, the adapter 6 may be configured to hold one, two, or three transducers. The adapter 6 may be configured to affix to the adhesive pad 5 by a fixed, swiveling or ratcheting connector 7. The adapter 6 may be configured to be detachable or interchangeable with a fixed, swiveling, or ratcheting connector 7. In some transducer embodiments, a swiveling or ratcheting connector 7 may facilitate patient position changes, e.g., by allowing the adapter 6 to swivel or ratchet relative to the adhesive pad 5. The adapter 6 may include fixed or adjustable rails 8, which may facilitate securing the pressure transducer to the adapter 6 on the adhesive backed pad 5. In some example embodiments, the adapter 6 may be configured with a clasp 9 with a pliable material 10, which closes across and molds to various manufacturer pressure transducers, which facilitates pressure transducer securement to the adapter 6. The clasp 9 with pliable material 10 is configured to remain attached to or be a molded part of the adapter 6, close across and deform to various shaped pressure transducers, and secure to the adapter clasp lock 11.

In preferred example embodiments, the adhesive back pad 5 may be made of, but not limited to, a lightweight dense tape, or a lightweight tape with fiberglass weaved throughout with a glue or an adhesive enabling securement to the phlebostatic axis 3 of the patient 1 (FIG. 1). The device elements, e.g., the connector 7, adapter 6, adapter rails 8, closing clasp 9, and adapter clasp lock 11, may be made of, but not limited to, a light weight plastic or composite material. The clasp pliable material 10, may be made of, but not limited to, lightweight foam, foam rubber, or a similar denser, pliable material.

The device elements are also shown in FIG. 3, which depicts a side, elevation view of the device including, but not limited to, a thin adhesive pad 5. In preferred example embodiments, the adhesive pad 5 of the device may be configured around or attached to a fixed, swiveling, or ratcheting connector 7. In some example embodiments, the adapter 6 may be affixed to, detachable or interchangeable with the device connector 7. The adapter 6 may have fixed or adjustable rails 8, which may facilitate securing the pressure transducer to the device via the adapter 6 on the adhesive back pad 5. In preferred example embodiments, the device adapter 6 may include a folding clasp 9 with a pliable material 10, which may close across and mold to various manufacturer pressure transducers, and secure to the adapter clasp lock 11 (FIG. 2), thus further facilitating pressure transducer securement to the device adapter 6.

In preferred embodiments, the adhesive back pad 5 may be made of, but not limited to, a lightweight dense tape, or a lightweight tape with fiberglass weaved throughout with a glue or an adhesive enabling securement to the phlebostatic axis 3 of the patient 1 (FIG. 1). The device elements, e.g., the connector 7, adapter 6, adapter rails 8, closing clasp 9, and adapter clasp lock 11, may be made of, but not limited to, a light weight plastic or composite material. The clasp pliable material 10 may be made of, but not limited to, light weight foam, foam rubber, or a similar denser, pliable material.

While preferred materials for elements have been described; the device is not limited by these materials. Composites, fiberglass, plastics, rubber, foam, tape, paper, glue, adhesives and other materials may include some or all of the elements of the pressure transducer securement device and apparatuses in various example embodiments of the present invention.

As may be seen from the above and with reference to FIGS. 1 through 3, a pressure transducer securing device may include an adhesive pad 5 that is securable to a patient 1 at a phlebostatic axis 3 of patient 1. An adapter 6 is coupled to adhesive pad 5. Adapter 6 is configured for receipt of a pressure transducer in order to mount the pressure transducer to patient 1. In various example embodiments, adapter 6 may be fixed or swivalable relative to adhesive pad 5.

Using the pressure transducer securing device, the pressure transducer may be secured to patient 1. For example, a healthcare provider may secure adhesive pad 5 to patient 1 at a phlebostatic axis 3 of patient 1. In particular, the healthcare provider may remove a backing from one side of adhesive pad 5 to expose the adhesive on adhesive pad 5. The healthcare provider may then position adhesive pad 5 on the patient 1 at a phlebostatic axis 3 of patient 1 such that the adhesive on adhesive pad 5, e.g., exposed by removing the backing, secures adhesive pad 5 to patient 1.

After securing adhesive pad 5 to patient 1, the pressure transducer may be slid into adapter 6 that is coupled to adhesive pad 5. For example, the healthcare provider may insert the pressure transducer into adapter 6 such that adapter 6 holds the pressure transducer on patient 1. When adapter 6 is swivalable relative to adhesive pad 5, the pressure transducer may rotate or pivot relative to patient 1. For example, when patient 1 moves, the pressure transducer may rotate or pivot to avoid discomfort caused by the pressure transducer tugging on patient 1. In particular, it will be understood that the pressure transducer may be connected to various lines, and the lines may tug or otherwise pull on patient 1 when patient 1 moves. By swiveling, discomfort to patient 1 may be advantageously reduced.

Adapter 6 may be removable from adhesive pad 5. By removing adapter 6 from adhesive pad 5, various (e.g., interchangeable) adapters 6 may be mounted to adhesive pad 5. In particular, a common adhesive pad 5 may be used with one or more adapter(s) 6 configured to support different pressure transducers, e.g., from various manufactures.

Adapter 6 may include a pair of flanges 12. The pressure transducer may be received between and/or under flanges 12 when the pressure transducer is received by adapter 6. Thus, flanges 12 may assist with securely mounting the pressure transducer to adapter 6. As noted above, after securing adhesive pad 5 to patient 1, the pressure transducer may be slid into adapter 6 that is coupled to adhesive pad 5. In particular, the healthcare worker may slide the pressure transducer between and/or under flanges 12 to mount the pressure transducer to adapter 6.

Adapter 6 may also include a clasp 9 configured to close across the pressure transducer to lock the pressure transducer onto adapter 6. For example, clasp 9 may close and extend across flanges 12. Clasp 9 may be a conforming or elastically deformable clasp configured to mold to a shape of the pressure transducer. Clasp 9 may assist with securely mounting the pressure transducer to adapter 6. In particular, clasp 9 may assist adapter 6 with securely supporting different pressure transducers, e.g., from various manufactures. As noted above, after securing adhesive pad 5 to patient 1, the pressure transducer may be slid into adapter 6 that is coupled to adhesive pad 5. In particular, the healthcare worker may slide the pressure transducer onto adapter 6, and the healthcare worker may close clasp 9 to lock the pressure transducer to adapter 6.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A pressure transducer securing device, comprising:
an adhesive pad securable to a patient at a phlebostatic axis of the patient; and
an adapter coupled to the adhesive pad, the adapter is configured for receipt of a pressure transducer in order to mount the pressure transducer to the patient,
wherein the adapter is fixed or swivalable relative to the adhesive pad.

2. The pressure transducer securing device of claim 1, wherein the adapter is removable from the adhesive pad.

3. The pressure transducer securing device of claim 1, wherein the adapter comprises a pair of flanges, the pressure transducer received between the pair of flanges when the pressure transducer is received by the adapter.

4. The pressure transducer securing device of claim 1, wherein the adapter comprises a clasp configured to close across the pressure transducer to lock the pressure transducer onto the adapter.

5. The pressure transducer securing device of claim 4, wherein the clasp is a conforming clasp configured to mold to a shape of the pressure transducer.

6. The pressure transducer securing device of claim 1, wherein the adhesive pad comprises a fiberglass weaved tape with an adhesive on one side of the fiberglass weaved tape.

7. A method for securing a pressure transducer to a patient, comprising:
securing an adhesive pad to the patient at a phlebostatic axis of the patient; and
sliding the pressure transducer into an adapter coupled to the adhesive pad,
wherein the adapter is fixed or swivalable relative to the adhesive pad.

8. The method of claim 7, wherein the adapter is removable from the adhesive pad.

9. The method of claim 7, wherein sliding the pressure transducer into the adapter comprises sliding the pressure transducer between a pair of flanges of the adapter.

10. The method of claim 7, further comprising closing a clasp of the adapter across the pressure transducer to lock the pressure transducer onto the adapter.

11. The method of claim 10, wherein the clasp molds to a shape of the pressure transducer.
